(19) United States
(12) Patent Application Publication
(10) Pub. No.: US 2006/0276808 A1
(43) Pub. Date: Dec. 7, 2006

(54) Minimally Invasive Methods and Apparatus for Accessing and Ligating Uterine Arteries with Sutures

(76) Inventors: Kevin R. Arnal, Excelsior, MN (US); James R. Mujwid, Crystal, MN (US)

Correspondence Address: AMS RESEARCH CORPORATION, 10700 BREN ROAD WEST, MINNETONKA, MN, 55343, US

(21) Appl. No.: 11/422,462

(22) Filed: Jun. 6, 2006

Related U.S. Application Data
(60) Provisional application No. 60/687,678, filed on Jun. 6, 2005.

(51) Int. Cl. A61B 17/04

(52) U.S. Cl. 606/148

(57) ABSTRACT

Surgical procedures, tools, and kits particularly suited to effect uterine artery ligation in a minimally invasive manner to treat uterine fibroids or other conditions of the uterus are disclosed. An incision through the fornix is made to access the uterine arteries supported by the cardinal and uterosacral ligaments. A distal end of one or more sutures is passed around each uterine artery and a portion of the ligament supporting and enclosing the uterine artery, wherein the portion may include a uterine vein. The passage is effected by directing a suture distal end along a first side of the portion of the ligament beyond to the fornix incision, then through the ligament, and then back proximally along the second ligament side toward the fornix incision.

The present application claims the benefit of U.S. Provisional Application No. 60/687,678 filed Jun. 6, 2005 (the entire contents incorporated by reference herein).

### FIELD

The present invention pertains to surgical procedures, tools, and kits particularly suited to effect uterine artery ligation in a minimally invasive manner to treat uterine fibroids or other conditions of the uterus.

### BACKGROUND

Each year, many women undergo a surgical removal of the uterus (hysterectomy) due to the growth of muscular tumors of the uterus (leiomyoma or uterine fibroids) or for uterine cancer, adenomyosis, menorrhagia, uterine prolapse, and dysfunctional uterine bleeding (abnormal menstrual bleeding that has no discrete anatomic explanation such as a tumor or growth). The uterus has a pear-shaped, uterine body extending between a fundus extending right and left to junctions with the right and left Fallopian tubes and a uterine neck (cervix) that extends to the vagina. The uterus has a smooth muscle uterine wall (myometrium) with an interior uterine mucosa (endometrium) that lines a uterine cavity extending between the right and left Fallopian tubes and a cervical opening of the cervix to the interior of the vagina. The uterine body is supported within the pelvis by right and left ligamentous structures such that the uterine body (fundus) is bent (anteflexed) and tilted (anteverted) anteriorly over the bladder and separated from the sacrum by the bowel. The uterine cervix extends into a tissue cul-de-sec of the vagina such that a flexible, annular trough (fornix) of the vagina surrounds and is integrally connected with the cervix.

The ligamentous structures each include a broad ligament (part of the peritoneum), a round ligament, an ovarian ligament, a uterosacral ligament, a cardinal ligament, and other tissue structures. The broad ligament is a broad fold of peritoneum extending over the uterus and from the lateral margins of the uterus to the wall of the pelvis; it is divided into the mesometrium, mesosalpinx, and mesovarium that extend on each lateral side of the uterus to the wall of the pelvis. The mesometrium is the portion of the broad ligament below the mesovarium, composed of the layers of peritoneum that separate to enclose the uterus. The mesovarium is a portion of the broad ligament of the uterus between the mesometrium and mesosalpinx, which is drawn out to enclose and hold the ovary in place. The uterosacral ligaments are parts of the thickening of the visceral pelvic fascia beside the cervix and vagina, passing posteriorly in the rectouterine fold to attach to the front of the sacrum. The cardinal ligaments are fibrous bands attached to the uterine cervix and to the vault of the lateral fornix of the vagina and are contiguous with the mesometrium sheathing the pelvic vessels including the right and left uterine arteries.

Oxygenated blood is provided to the normal uterine cells and the cells of uterine fibroids by a redundant arterial blood supply denoted in the bilateral left and right uterine arteries and the bilateral left and right ovarian arteries. The right and left uterine arteries branch from the internal iliac artery and cross over the ureter at the level of the internal ostium (os) of the cervix and each divides into ascending and descending limbs. The ascending limb runs tortuously upward, between the leaves of the broad ligament, and supplies horizontal anterior and posterior branches to the cervix and the corpus. The descending branch of the uterine artery turns inferiorly and supplies the vagina from the lateral aspect. The ascending and descending branches of the uterine arteries extend through and are enclosed within the cardinal ligaments.

Uterine leiomyomas, commonly known as fibroids or myomas, are well circumscribed, solid, benign tumors arising from the smooth muscle of the myometrium, and are composed of smooth muscle cells and extracellular matrix. Fibroids may occur in several locations within the uterine wall and are named subserosal, submucosal, or intramural depending on their location. Subserosal leiomyomas are located just under the uterine serosa and may be attached to the corpus by a narrow or a broad base. Intramural leiomyomas are found predominantly within the thick myometrium and may distort the uterine cavity or cause an irregular external uterine contour. Submucous leiomyomas are located within the myometrium proximate to the endometrium. Uterine leiomyomas or fibroids are clinically apparent in 20% to 25% of women during the reproductive years and cause symptoms necessitating treatment, typically surgical removal of the uterus.

The surgical removal of the uterus requires exposing it sufficiently, ligating and severing the arteries and Fallopian tubes, severing the broad ligament and other ligaments from the uterine body, and severing the cervix from the fornix. Thus, in addition to the loss of reproductive capability, a hysterectomy requires major invasive surgery that can involve excessive blood loss, prolonged convalescence, attendant pain and discomfort, and economic costs. Newer treatment methods have been developed or proposed for at least some of these diseases and conditions. Nevertheless, hysterectomy remains the treatment of choice to treat the conditions and diseases listed above while less drastic treatments continue to be explored.

In the case of uterine fibroids, intraluminal occlusion of the right and left uterine arteries has been demonstrated as efficacious in starving and killing fibroid cells in situ while leaving normal uterine cells intact. For example, uterine artery occlusion or embolization was demonstrated as effective in eliminating or lessening uterine fibroids in Ravina et al., “Arterial Embolization to Treat Uterine Myomata”, Lancet, 1995; Vol. 344; pp. 671-692. In this technique, uterine arteries are accessed via a trans-vascular route from a common femoral artery disposing a delivery catheter within the left and right uterine arteries, and embolic coils are dispensed from the catheter into the uterine arteries to promote clotting and thereby occlude the arterial passageways. When the uterine arteries are occluded in this fashion (or in any other fashion), the normal uterine cells and the fibroid cells of fibroids within the fundus are deprived of one blood supply. However, as demonstrated by Ravina et al., the effect on the fibroid cells is greater than the effect on normal uterine cells. In most instances, the relatively faster growing fibroid cells require a higher volume of blood oxygen and die when starved of oxygen, leading to fibroid shrinkage and cessation or diminution of clinical symptoms. Various methods of intraluminal occlusion of the right and left uterine arteries near the upper and lower branches thereof are also set forth in U.S. Pat. No. 6,602,251 and in U.S. Patent Application Publication No. 2004/0202694, for example.

Such catheter-based uterine artery embolization must be performed with fluoroscopic or other visualization equipment by an interventional radiologist trained in catheterization and embolization delivery techniques. Thus, other invasive or non-invasive or minimally invasive techniques have been proposed and clinically explored to access the bilateral uterine arteries trans-vaginally or in a laparoscopic approach from a skin incision to temporarily or permanently apply compressive force around and thereby close the uterine arteries.

For example, trans-vaginal uterine artery occlusion with sutures tied around the right and left uterine ligaments exposed via a cervical incision is described by Harmanli, M D et al., in “Trans-vaginal Uterine Artery Ligation in a Woman with Uterine Leiomyomas”, Journal of Reproductive Medicine, May 2003, Vol. 48; pp. 384-386. In this minimally invasive approach through the vagina, the uterosacral and cardinal ligaments are exposed by an annular incision around of the cervix, and the sutures are tied around the right and left uterine arteries and the supporting ligaments to occlude the arteries. Preferably, sutures are tied around the right and left uterosacral ligaments supporting descending branches of the uterine arteries and at least a proximal portion of the right and left cardinal ligaments supporting ascending branches of the uterine arteries. Thus, it is necessary to surgically expose both ligaments in a trans-vaginal procedure that is customarily followed in the initial steps of performing a hysterectomy. Typically in a hysterectomy, the uterine arteries are first clamped, ligated, or cauterized to halt blood flow to the uterus before the supporting ligaments are severed along the uterine wall.

A wide variety of further uterine artery occlusion techniques have been proposed in U.S. Pat. Nos. 6,254,601 and 6,546,933, for example, and in Burbank, et al., “Uterine Artery Occlusion by Embolization or Surgery for the Treatment of Fibroids: A Unifying Hypothesis-Transient Uterine Ischemia,” The Journal of the American Association of Gynecologic Laparoscopists, November 2000, Vol. 7, No. 4 Supplement, pp. S3-S49. In particular, various methods and apparatus have been suggested to determine the location of the uterine arteries, effect either permanent of temporary cessation of blood flow through the uterine arteries to the uterus to starve uterine fibroids of sufficient oxygenated blood, and to verify the blood flow cessation.

Various additional tools and minimally invasive techniques are presented for applying permanent or resorbable sutures or occlusion devices including snares, clips, and clamps, about at least a portion of each cardinal ligament and uterine artery are disclosed in U.S. Pat. Nos. 6,506,156, 6,550,482, 6,602,251, 6,635,065, and 6,638,286 and in U.S. Patent Application Publication Nos. 2002/0124853 and 2003/0120286. Typically, access to the uterine arteries and cardinal ligaments is obtained by instruments introduced trans-vaginally to make one or more incision through the fornix and to advance and affix the suture or occlusion device about a portion of each cardinal ligament and uterine artery tightly enough to diminish or halt oxygenated blood flow.

Furthermore, tools adapted to be trans-vaginally applied temporarily to occlude the right and left uterine arteries are disclosed in U.S. Patent Application Publication Nos. 2002/0124853, 2002/0165579, 2002/0183771, 2003/0120306, 2003/0191391, 2004/0097961, 2004/0092979, 2004/0097962, 2004/0153105, 2004/0158262, and 2005/0113852. In one approach, the distal end of the tool that is advanced into the vagina is advanced into the fornix alongside the cervix to stretch the fornix and compress the right and/or left uterine artery against itself or against the cervix. The tool distal end is not advanced through the wall of the fornix, and the approach is therefore characterized as non-invasive. The tool may include a blood flow sensor to assist in disposing the tool distal end against a uterine artery and to verify that blood flow is reduced or halted when the uterine artery is compressed. The tool is applied to the uterine artery or arteries for a sufficiently long time period to effect starvation and death of uterine fibroids and is then withdrawn, allowing oxygenated blood flow to be restored unless occlusion has taken place.

Despite these approaches, it would be desirable to provide improved surgical instruments, tools and/or occluding devices and procedures that can be safely, simply, and readily employed to effect temporary or permanent occlusion of the uterine arteries, particularly through a non-invasive or minimally invasive trans-vaginal approach. Since their gynecologist diagnoses the majority of the patients suffering from fibroids, there is an advantage to the patient to be treated with a procedure that can be performed by their gynecologists. Because of the perceived risk of patient loss to referral, many patients will go untreated or are recommended a hysterectomy to treat their symptoms. If a trans-vaginal approach that follows many of the steps of a hysterectomy, which most gynecologist can perform, were available for the treatment of fibroids, many more patients would benefit from the relief of their symptoms with a minimally invasive surgery that could be performed by their gynecologist.

### SUMMARY

The preferred embodiments of the present invention incorporate a number of inventive features that address the above-described needs that may be combined as illustrated by the preferred embodiments or advantageously separately employed. The surgical procedures, tools and kits of the present invention are not necessarily limited to but are particularly suited to effect uterine artery ligation in a minimally invasive manner to treat uterine fibroids or other conditions of the uterus by diminishing or blocking arterial blood flow to treat uterine diseases or disorders.

The surgical procedures, tools and kits of the preferred embodiments of the present invention may be employed to restrict or block uterine arterial blood flow to treat uterine fibroids or other conditions through a minimally invasive trans-vaginal approach placing one or more sutures around the uterine arteries and at least a portion of the ligaments surrounding the uterine arteries.

The methods of the preferred embodiments of the present invention involve making incisions through the fornix to access the uterine arteries supported by the cardinal and uterosacral ligaments. A distal end of one or more sutures is passed around each uterine artery and a portion of the ligament supporting and enclosing the uterine artery, wherein the portion may include a uterine vein. The passage is effected by directing a suture distal end along a first side of a portion of the ligament beyond the fornix incision, then through the ligament, and then back proximally along the second ligament side toward the fornix incision.

In preferred embodiments, a suture delivery instrument engages the suture to present the suture distal end substantially at the distal end of the suture delivery instrument. The distal sections of the suture and the suture delivery instrument are advanced along a first ligament side, and the suture delivery instrument is manipulated to advance the suture distal end through the ligament to the second ligament side distal to the fornix incision. The suture distal end is engaged and retracted proximally along the second ligament side and the suture delivery instrument is retracted proximally, whereby segments of the suture extend around and along the sides of the portion of the ligament distal to the fornix incision. The suture proximal and distal ends are then grasped and tensioned to close the uterine artery, a suture knot is tied, the suture free ends are trimmed, the cessation of blood flow is ascertained, and the incision is closed. The procedure may be repeated if the blood flow is insufficiently blocked.

Preferably, the suture distal end is coupled to a tissue-penetrating tip, e.g., a needle or barb or hook or cone or dart, (hereinafter referred to as a “tissue-penetrating barb” that facilitates passage through ligamentous tissue supporting the uterine artery so that the suture distal end can be engaged on the second ligament side beyond the fornix incision and withdrawn proximally. For convenience, the suture coupled to a tissue-penetrating barb is referred to herein as a suture assembly.

Moreover, the suture delivery tools and surgical procedures of the preferred embodiments involve use of a suture snare that may be part of or affixed to the suture delivery instrument proximal end. The suture snare can be applied to engage the tissue-penetrating barb or suture distal end to be retracted proximally along the first ligament side, whereby segments of the suture extend around and along the sides of the portion of the ligament beyond the fornix incision. Preferably, a positive engagement of the tissue-penetrating barbs with the suture snare is made, and the engagement is audibly or otherwise conveyed to the physician operating the suture delivery instrument.

Preferably, the suture snare is detachable from the suture delivery instrument body proximal end, and the suture extends through at least a portion of both the suture snare and the suture delivery instrument body. A length of the suture is preferably stored within a cavity of the detachable suture snare body or a cavity of the suture delivery instrument body. In use, the suture snare is manually detached from the suture delivery instrument body and advanced along the second ligament side to make engagement with the suture distal end.

Preferably, the engagement is effected by passage of a tissue-penetrating barb through a membrane supported by the snare body. The membrane penetration is advantageously audible. Moreover, preferably, the membrane supports a suture loop at the suture proximal end, and the tissue-penetrating barb passes into the suture loop when it penetrates the membrane. The suture loop is thereafter preferably detachable from the membrane and can be drawn tight around the suture at or near the suture distal end.

In any of these embodiments, a suture passage slot may be provided extending laterally from the lumen of the suture delivery instrument body to facilitate detachment of the suture delivery instrument from the suture. Similarly, a suture passage slot may be provided extending laterally from the cavity of the snare body or the delivery instrument body to facilitate detachment of the suture therefrom.

Preferably, the suture is a conventional resorbable or bioabsorbable suture that is absorbed by the body after a time period sufficient to kill or diminish uterine fibroids with or without achieving total occlusion of the uterine arteries. Thus, the methods, tools and/or components of the preferred embodiments of the present invention occlude one uterine artery or both uterine arteries on a chronic or temporary basis.

Advantageously, procedures for occluding uterine arteries are simplified through the use of the instruments of the present invention to shorten the surgical time and trauma to the patient. The instruments of the present invention may be advantageously employed in ligating uterine arteries exposed through a conventional trans-vaginal surgical approach surrounding the cervix that is well known to and practiced by many surgeons. Moreover, it is not necessary in the practice of the present invention to employ instruments or equipment for locating the uterine arteries and verifying successful occlusion of the uterine arteries blocking blood flow.

This summary of the invention has been presented here simply to point out some of the ways that the invention overcomes difficulties presented in the prior art and to distinguish the invention from the prior art and is not intended to operate in any manner as a limitation on the interpretation of claims that are presented initially in the patent application and that are ultimately granted.

### BRIEF DESCRIPTION OF THE DRAWINGS

These and other advantages and features of the present invention will be more readily understood from the following detailed description of the preferred embodiments thereof, when considered in conjunction with the drawings, in which like reference numerals indicate identical structures throughout the several views, and wherein:

FIG. 1 is a schematic illustration of a first embodiment of a suture suitable terminating in a distal tissue-penetrating barb for ligating a uterine artery employing a suture delivery tool of the present invention;

FIG. 2 is a schematic illustration of a second embodiment of a suture terminating in a distal tissue-penetrating barb suitable for ligating a uterine artery employing a suture delivery tool of the present invention;

FIG. 3 is a schematic illustration of a third embodiment of a suture suitable terminating in a distal tissue-penetrating barb for ligating a uterine artery employing a suture delivery tool of the present invention;

FIG. 4 is a plan view of a distal portion of a first embodiment of a suture delivery tool of the present invention;

FIG. 5 is a plan view of the distal portion of the first embodiment of a suture delivery tool of the present invention loaded with a suture of FIG. 3;

FIG. 6 is a cross-section view of the distal portion of the first embodiment of the suture delivery tool loaded with a suture of FIG. 5 disposed in relation to a uterine artery supported by a ligament with the tissue-penetrating barb passed through the ligament;

FIG. 7 is a plan view of a suture snare engaging the tissue-penetrating barb to draw the suture around both sides of the ligament;

FIG. 8 is a plan view of the use of the distal portion of the suture delivery tool to capture the tissue-penetrating barb to draw the suture around both sides of the ligament;

FIG. 9 is a plan view of a second embodiment of a suture delivery tool of the present invention formed having an interlocking proximal suture snare and a distal suture delivery instrument and loaded with a suture of FIG. 1, for example;

FIG. 10 is a plan view of suture delivery tool of FIG. 9 loaded with a suture of FIG. 1 with the proximal snare detached from the distal suture delivery instrument;

FIG. 11 is a plan view of the suture delivery tool of FIG. 9 disposed in relation to a uterine artery supported by a ligament with the tissue-penetrating barb passed through the ligament;

FIG. 12 is a plan view of the suture snare of the suture delivery tool of FIG. 9 detached from the suture delivery instrument and employed to capture the tissue-penetrating barb to withdraw the suture around both sides of the ligament and proximally;

FIG. 13 is an expanded detail perspective view of the suture delivery instrument of FIG. 9 depicting an optional suture release slit;

FIG. 14 is an expanded detail view in partial cross-section depicting a preferred support of a proximal end suture loop by a penetrable membrane and storage of a proximal portion of the suture in a snare body cavity;

FIG. 15 is a plan view of a variation of the second embodiment of a suture delivery tool of the present invention formed having an interlocking proximal suture snare and a distal suture delivery instrument and loaded with a suture of FIG. 1, for example;

FIG. 16 is a plan view of suture delivery tool of FIG. 15 loaded with a suture of FIG. 1 with the proximal snare detached from the distal suture delivery instrument;

FIG. 17 is an expanded partial perspective view of the distal portion of the suture delivery instrument of the suture delivery tool of FIG. 15;

FIG. 18 is an expanded partial perspective view of the snare membrane of the suture snare of the suture delivery tool of FIG. 15 supporting a suture loop;

FIG. 19 is a schematic illustration of a tissue-penetrating barb extending through a suture loop of a suture extending around a ligament supporting a uterine artery, for example; and

FIG. 20 is a schematic illustration of the suture loop tightened about the suture body and the suture being drawn tight to ligate the uterine artery.

It will be understood that the drawing figures are not necessarily to scale.

### DETAILED DESCRIPTION

In the following detailed description, references are made to illustrative embodiments of methods and apparatus for carrying out the invention. It is understood that other embodiments can be utilized without departing from the scope of the invention. The surgical procedures, tools and kits of the present invention are not necessarily limited to but are particularly suited to effect uterine artery ligation in a minimally invasive manner to treat uterine fibroids or other conditions of the uterus. Thus, preferred methods and apparatus are described for controlling uterine arterial blood flow by diminishing or blocking arterial blood flow to the uterus to treat diseases and disorders, e.g., uterine fibroids and uterine bleeding.

The methods of the preferred embodiments of the present invention involve making incisions through the fornix to access the uterine arteries and cardinal ligaments. One suitable approach to accessing the right and left uterosacral and cardinal ligaments supporting the uterine arteries is described in the above-referenced Harmanli M D et al publication.

The preferred embodiments provide for the passage of a distal end of one or more suture around each uterine artery and a portion of the ligaments supporting and enclosing the uterine artery, e.g. around the cardinal ligament and at least a portion of the uterosacral ligament supporting the ascending and descending branches, respectively. The accessed portion of the cardinal ligament is shaped like a band having first and second ligament sides. The passage of the suture is effected by directing a distal end of the suture along one side of the portion of the ligament beyond the fornix incision, then through the ligament, and then back proximally toward the fornix incision. In this way, a single suture may be passed around a portion of the cardinal ligament and the uterosacral ligament.

Advantageously, the surgical approach to the ligaments supporting the uterine arteries renders the ligaments visible and severs arterial vessels branching from the uterine arteries. It is well known that the exposed ligaments support the ascending and descending branches of the uterine arteries (even if the uterine arteries cannot be seen). Therefore, it is not necessary to employ vessel detection or imaging equipment or instruments, e.g., arterial blood flow detection by Doppler sensing or the like or by fluoroscopic imaging equipment or instruments or the like. The successful occlusion of the uterine arteries can sometimes be verified by simply visually observing a reduction or cessation of arterial blood bleeding along the cervical incision as the suture is tied around the ligament or ligaments supporting the uterine arteries. However, blood flow sensing techniques described above could be utilized to verify arterial occlusion, if desired.

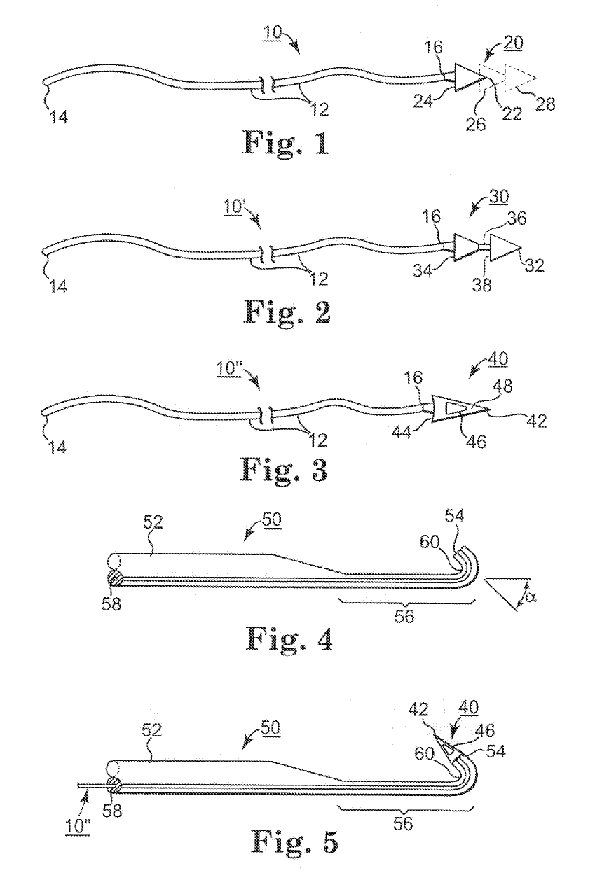

Preferably, the sutures employed in the practice of the present invention are formed having a tissue-penetrating barb, e.g., a barb or needle or hook or cone or dart, at the suture distal end that penetrates ligamentous tissue when the suture distal end is passed around the uterine artery and through a selected portion of the ligament. The tissue-penetrating barb is shaped to penetrate by cutting or separating ligamentous tissue fibers and is further shaped to inhibit retraction back through the puncture through the ligament. Either the tissue-penetrating barb or the suture distal end coupled to the tissue-penetrating barb can be engaged to be retracted proximally on the other side of the portion of the ligament. In reference to FIGS. 1-3, exemplary suture assemblies 10, 10′ and 10″, respectively, depict alternative distal tissue-penetrating barbs 20, 30, 40.

Each suture assembly 10. 10′, 10″ comprises a length of suture 12 extending between a suture proximal end 14 and a suture distal end 16. The suture 12 may be formed of a permanent suture material or a resorbable suture material of any of the types conventionally employed in surgery and/or disclosed in certain of the referenced patents and publications. The length of suture 12 may be selected to enable passage around a portion of a uterine artery supported by a ligament, e.g., the uterosacral and/or cardinal ligament, and tied off. The tissue-penetrating barb 20, 30, 40 are intended to be severed from suture 12 before or after tying of the suture 12.

In FIG. 1, the tissue-penetrating barb 20 is formed as a generally conical or arrow shaped dart extending proximally from a distal tissue-penetrating point 22 to a proximal shoulder 24 coupled in any suitable fashion with suture distal end 16. The tissue-penetrating barb 20 may be formed of a series of such darts aligned axially, e.g., darts 26 and 28 depicted in broken lines extending distally from sharp point 22, to provide a serrated profile. A conical dart has a substantially circular shoulder 24, whereas an arrow-shaped dart has a substantially elliptical shoulder 24. In each case, the widened shoulder 24 (or shoulders) positively engages the ligament and inhibits retraction through the tissue puncture formed when the tissue-penetrating barb is passed through ligamentous tissue. Preferably, the shoulder 24 is a rim of a concave proximal end of the tissue-penetrating barb to enhance its retention power, and the suture distal end 16 is attached distal to the rim. Moreover, the distal tissue-penetrating point 22 may either be sharp tip effect tissue cutting during penetration or somewhat blunt and shaped to effect tissue separation during penetration.

In a variation depicted in FIG. 2, the tissue-penetrating barb 30 is formed as a generally conical or arrow-shaped dart extending proximally from a distal tissue-penetrating point 32 to a proximal shoulder 38 that is in turn coupled by a reduced diameter neck 36 to a second such dart extending proximally to a shoulder 34. The suture distal end 16 is coupled to the shoulder 34 in any suitable fashion.

In a further variation depicted in FIG. 3, the tissue-penetrating barb 40 is formed as a generally conical or arrow-shaped dart extending proximally from a distal tissue-penetrating point 42 to a proximal shoulder 44 coupled in any suitable fashion with suture distal end 16. The tissue-penetrating barb 40 may generally conform to any of the shapes of tissue-penetrating barb 20 except that a feature or hole 46 extends laterally through the suture dart 48 that can be snared as described further below.

A first preferred embodiment of a suture delivery instrument 50 is depicted in FIGS. 4 and 5 that comprises an elongated instrument body 52 extending from a proximal end (not shown) adapted to be grasped by the surgeon and a distal end 54 adapted to be applied alongside and against one side of a ligament supporting the right or left uterine artery. The instrument body 52 may be circular or substantially oval or rectangular in cross-section through most of its length having a configuration and cross-section area facilitating handling and providing sufficient column strength to be advanced trans-vaginally under visualization through the incision. A distal portion 56 of the instrument body 52 is preferably reduced in cross-section area and bent at an angle α that may be 90° or less to present the instrument body distal end 54 toward a side of the ligament. A suture lumen 58 extends from a proximal lumen opening (not shown) at or distal to the instrument body proximal end (not shown) distally through the instrument body 52 to a distal lumen opening at instrument body distal end 54. It will be understood that the suture lumen proximal end opening may be located at any point along the instrument body 52, e.g., at the bend in the distal portion 56, whereby the suture 12 may extend alongside a minor or substantial portion of the instrument body 52. The instrument body 52 may be simplified in fabrication and minimized in cross-section area without sacrificing rigidity necessary to transmit tissue penetrating force to a tissue-penetrating barb. Moreover, the installation of the suture 12 through the suture lumen 58 in the surgical field may also be made easier if the proximal lumen opening is located at the bend in the distal portion 56.

One of the suture assemblies 10, 10′, 10″ or the like may be supported by the suture delivery instrument 50, e.g., suture assembly 10″ depicted in FIG. 5. At least a distal portion of suture 12 extends through the suture lumen 58 such that the tissue-penetrating barb 40 is disposed extending from the instrument body distal end 54. The enlarged shoulder 44 abuts the instrument body distal end 54 so that tissue-penetrating barb 40 cannot be drawn or retracted into suture lumen 58.

In use, the surgeon may grasp the instrument body proximal end to advance the distal portion 56 of the instrument body 52 with suture assembly 10″ loaded in this fashion into suture lumen 58 through the incision in the fornix and along one side of the exposed ligament supporting the right or left uterine artery. It may be desirable to rotate the instrument body proximal end to rotate the distal portion 56 of the instrument body 52 with suture assembly 10″ loaded in this fashion 90° in order to extend the instrument body distal end 54 and tissue-penetrating barb 40 alongside and past the uterine artery supported within the ligament without snagging tissue with the tissue-penetrating point 42. The instrument body distal end 54 and tissue-penetrating barb 40 may then be disposed alongside and past the uterine artery supported within the ligament.

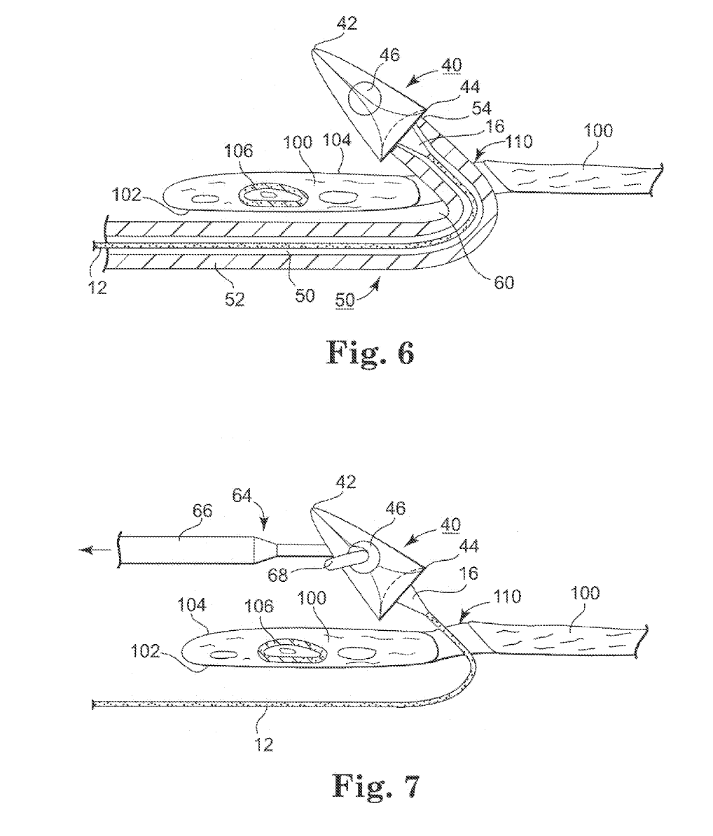

The cardinal and/or uterosacral ligament 100 and uterine artery 106 disposed between the first and second ligament sides 102 and 104 are depicted in FIGS. 6-8. As shown in FIG. 6, the tissue-penetrating barb 40 is advanced through the ligament 100 after a suitable position is reached along the first ligament side 102 to dispose the instrument body distal end 54 and tissue-penetrating barb 40 on the second ligament side 104. In this process, the tissue-penetrating point 42 is advanced past the uterine artery 106 and oriented toward the first ligament side 102. The delivery instrument body 52 is then retracted slightly so that the tissue-penetrating point 42 hooks into and penetrates through the ligament 100. The penetration dilates the ligament 100 to form puncture 110, and continued retraction of the instrument body 52 enables passage of the instrument body distal end 54 through the puncture 110 until the notch 60 bears against the puncture 110.

At this point, it is necessary to remove the suture delivery instrument 50 over the suture 12, leaving the tissue-penetrating barb 40 along the second ligament side 104 and the suture 12 extending proximally along the first ligament side 102 as shown in FIG. 7. The suture delivery instrument 50 may be advanced past the puncture 110 to urge the instrument body distal end 54 out of the puncture 110 disposing it along the first ligament side 102. The suture delivery instrument 50 may then be retracted proximally along the first ligament side 102.

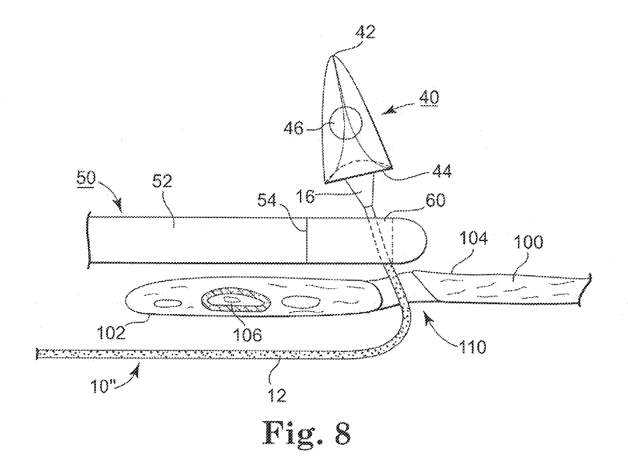

Subsequently, the suture distal end 16 or the tissue-penetrating barb 40 can be engaged and retracted proximally along the second ligament side 104 through use of a suture snare 64 as depicted in FIG. 7 or the suture delivery instrument 50 itself as depicted in FIG. 8.

With respect to FIG. 7, the distal hook 68 of the snare 64 can be advanced along the second ligament side 104 and through the hole 46 to engage the tissue-penetrating barb 40 by manipulation of the snare handle 66. The distal hook 68 could alternatively be applied around the suture 12 near the suture distal end 16, whereby the snare 64 may be employed with any of the suture assemblies 10, 10′ and 10″. It will be understood that the snare 64 may be alternatively be used in this same manner to engage the tissue-penetrating barb 40 or the suture distal end 16 while the suture assembly 10″ is still supported by the suture delivery instrument 50 and positioned as depicted in FIG. 6.

The snare handle 66 is then retracted proximally from the vagina to draw the suture 12 through the puncture 110 and proximally along the second ligament side 104. The suture delivery instrument 50 may also be retracted proximally over the suture 12 along the first ligament side 102 if the suture delivery tool is in the position depicted in FIG. 6 while the snare handle 66 is retracted.

With respect to FIG. 8, the notch 60 of suture delivery instrument 50 may itself be used as a snare to engage the suture 12 near the suture distal end 16, whereby the notch 60 may be employed with any of the suture assemblies 10, 10′ and 10″. The suture delivery instrument 50 is first withdrawn proximally over the suture 12 as described above and then advanced distally along the second ligament side 104 to position the notch 60 to tissue-penetrating barb the suture 12 at about the suture distal end 16. Then, the suture delivery instrument 50 may be retracted proximally along first ligament side 102 to draw the suture 12 and tissue-penetrating barb 40 proximally along the first ligament side 102.

In any of these ways, the suture 12 then extends through puncture 110 around and along the first ligament and second sides 102 and 104, and the proximal and distal ends 14 and 16 are made accessible to the surgeon. The tissue-penetrating barb 40 may be detached from the suture 12. The surgeon may then manually grasp or use suture gripping instruments to grip and tension the suture 12 around the portion of the ligament 100 supporting the uterine artery 106 to obstruct blood flow. The cessation of blood flow may be checked by observing whether bleeding about the cervical incision has ceased or lessened, and a suture knot may be tied and the suture free ends may be trimmed if cessation is verified. The procedure may be repeated to tie an additional suture around the uterine artery if the arterial blood flow is insufficiently blocked.

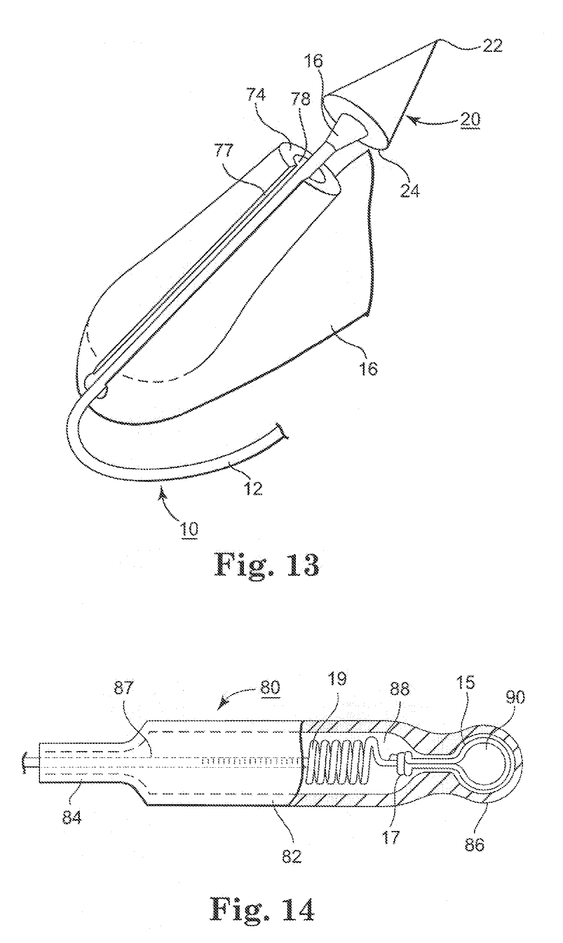

Turning to FIGS. 9-12, a suture delivery tool of the invention is depicted that provides additional advantages and features, in that it combines the suture delivery instrument 70 and a suture snare 80 and integrates, for ease of handling, any suture having a distal tissue-penetrating barb, e.g., the depicted suture assembly 10 having distal barb 20. The suture delivery instrument 70 detachably supports the suture snare 80 with the suture 12 extending therebetween. Moreover, the snare 80 provides the surgeon with positive audible and tactile feedback of capture of the tissue-penetrating barb 20 by the snare 80. Additional optional features of this suture delivery tool are depicted in FIGS. 13 and 14.

The suture delivery instrument 70 comprises an elongated delivery instrument body 72 extending from a delivery instrument proximal end 79 to a distal end 74. Again, the instrument body 72 may be circular or substantially oval or rectangular in cross-section through most of its length having a configuration and cross-section area facilitating handling and providing sufficient column strength to be advanced trans-vaginally under visualization through the incision. A distal body portion 76 of the instrument body 72 is again preferably reduced in cross-section area and bent at an angle α that may be 90° or less to present the instrument body distal end 74 toward a side of the ligament. A suture lumen 78 extends from a proximal lumen opening within instrument proximal end 79 all the way through the instrument body 72 to a distal lumen opening at instrument body distal end 74 or alongside at least a part of instrument body 72, e.g., in the straight section of the distal body portion 76 as shown in FIGS. 9-12. It will be understood that the suture lumen 78 may alternatively extend through the full length of the instrument body 72 as illustrated and described above with respect to the suture delivery instrument 50.

One of the suture assemblies 10, 10′, 10″ or the like may be supported by the suture delivery instrument 70, e.g., suture assembly 10 depicted in FIGS. 9-12. At least a distal portion of suture 12 extends through the suture lumen 78 such that the tissue-penetrating barb 20 is disposed extending from the instrument body distal end 74. The enlarged shoulder 24 abuts the instrument body distal end 74 so that tissue-penetrating barb 20 cannot be drawn into suture lumen 78 and the sharp point 22 is extended distally from the instrument body distal end 74 to penetrate tissue.

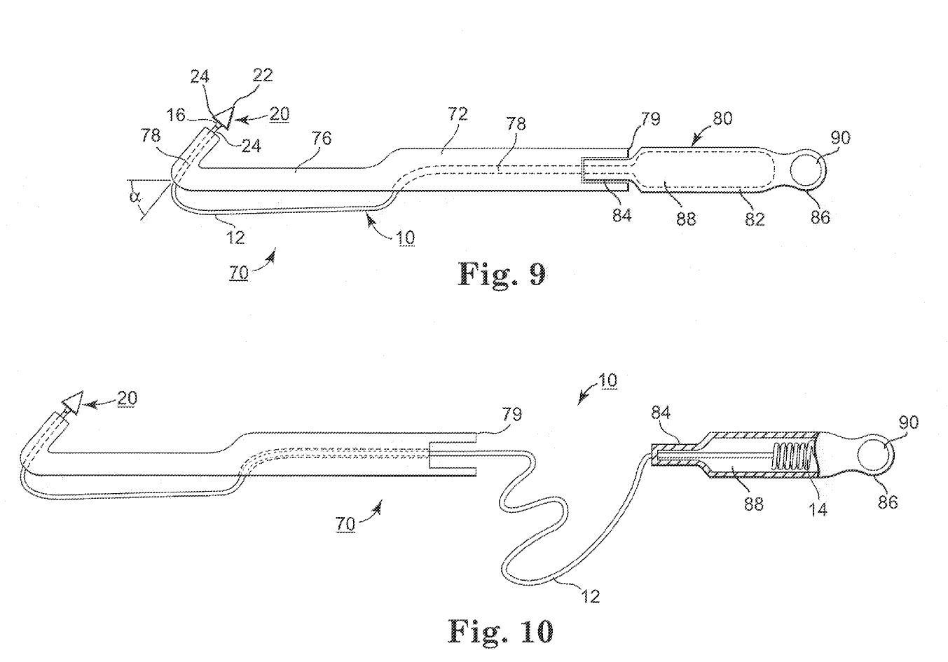

The snare 80 is formed of a snare body extending from a snare distal end 84 to a ring-shaped snare proximal end 86. The snare distal end 84 is shaped to be detachably coupled to the delivery instrument proximal end 79 employing any suitable snap or threaded coupling mechanism or interlock. The suture delivery instrument 70 and snare 80 are provided in sterile packaging attached together and with the suture assembly 10 loaded as depicted in FIG. 9. The bulk of the suture 12 is loaded into a snare cavity 88 (shown in FIG. 10) within snare body 82 during assembly of the suture 10, the snare 80, and suture delivery instrument 70. The suture proximal end 14 may be permanently attached to the snare 80 within the snare cavity 88 during such assembly. The suture 12 extends through an axial bore of the snare distal end 84 that is aligned with the proximal lumen end opening of suture lumen 78 when the snare 80 attached to the suture delivery instrument 70 as shown in FIG. 9. An intermediate length of the suture 12 can be drawn out of the snare cavity 88 through the axial bore of the snare distal end 84 when the snare 80 is detached and separated apart from the suture delivery instrument 70 as shown in FIG. 10.

The suture delivery instrument 70 and snare 80 are adapted to be grasped by the surgeon when attached together as shown in FIG. 9 to apply the distal end 74 and tissue-penetrating dart 20 alongside and against one side of a ligament supporting the right or left uterine artery. Thus, in use, the surgeon may grasp a proximal portion of the instrument body 72 and the snare body 82 to advance the distal body portion 76 of the instrument body 72 with suture assembly 10 loaded as described above through the incision in the fornix and along one side of the exposed ligament supporting the right or left uterine artery. During advancement, it may be desirable to rotate the assembled instrument body 72 and snare body 82 to thereby rotate the distal body portion 76 of the instrument body 72 (with suture assembly 10 loaded as depicted) 90° in order to avoid snagging tissue with the tissue-penetrating point 22. The instrument body distal end 74 and tissue-penetrating barb 20 may then be disposed alongside and past the uterine artery supported within the ligament.

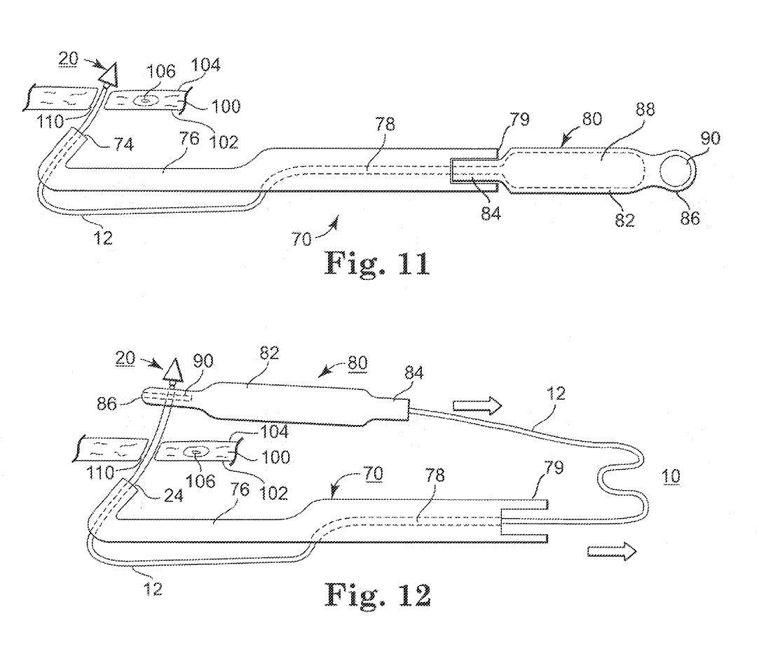

For example, the cardinal and/or uterosacral ligament 100 and uterine artery 106 disposed between the first and second ligament sides 102 and 104 are depicted in FIGS. 11 and 12. As shown in FIG. 11, the tissue-penetrating barb 20 punctures and is advanced through the puncture 110 of ligament 100 after a suitable position is reached along the first ligament side 102. The tissue-penetrating barb 20 is disposed on the second ligament side 104, and the instrument body distal end 74 is retracted in the manner described above with respect to the passage of tissue-penetrating barb 40 through puncture 110 depicted in FIGS. 6-8.

At this point, or while the instrument body distal end 74 still extends through the puncture 110 and supports the tissue penetrating point 20, the suture distal end 16 proximal to the shoulder 24 can be engaged and retracted proximally along the second ligament side 104 through use of snare 80 as depicted in FIG. 12.

The snare proximal end 86 is formed in a ring shape supporting a taut membrane 90 that can be penetrated by the tissue-penetrating point 22. The taut membrane 90 may be formed of a thin plastic material that is mechanically attached to or is formed integrally with the snare proximal end. In either case, the membrane 90 is relatively tough and tensioned like a drum head, though readily penetrable by the distal tissue-penetrating point 22 and capable of being stretched. Suitable materials for fabricating membrane 90 include polyurethane, polyethylene, natural rubber or silicone rubber film membranes. The membrane diameter exceeds the diameter or width of the shoulder 24 so that the entire tissue-penetrating barb 20 may be passed through the membrane 90.

As shown in FIG. 12, the snare 80 is detached from the suture delivery instrument 70 and reversed end for end as suture 12 is dispensed from the snare cavity 88. The snare proximal end 86 is then advanced through the vagina and the incision distally alongside the second ligament side 104 until the membrane 90 is disposed against the tissue penetrating point 22. Force is applied through the snare 80 to press the membrane 90 against the tissue penetrating point 22 until the tissue-penetrating barb 20 perforates the membrane 90. As noted above, it may be desirable to simultaneously apply a counter force through the suture delivery instrument 70 to hold the tissue-penetrating barb 20 steady during penetration through membrane 90.

The suture distal end 16 is snared by membrane 90 and the ring-shaped snare proximal end 86 as force is applied laterally to retract the snare 80. Thus, the snare 80 and the tissue-penetrating barb 20 may be retracted proximally along second ligament side 104 and through the vagina thereby drawing the suture 12 through the puncture 110 and proximally along the second ligament side 104. The suture delivery instrument 70 may also be simultaneously retracted proximally along the first ligament side 102 over the suture 12 as shown in FIG. 12. During this process, the suture 12 is drawn out of the snare cavity 88.

It will be understood that the advantages of the above-described embodiment of FIGS. 9-12 may alternatively be achieved in a suture delivery tool where the suture snare 80 is not detachable from the instrument body proximal end. In such a variation, the combined suture delivery tool would be retracted along the first ligament side 102 proximally out of the vagina and reversed end for end. The membrane 90 at the snare proximal end 86 would then be advanced trans-vaginally, through the cervical incision, and along the second ligament side 104 to effect snaring and retraction of the suture distal end 16 as described above. A length of the suture 12 may or may not be stored within a cavity of the tool body of the combined suture delivery tool.

Retraction of the suture delivery instrument 70 and release of the suture 12 may be facilitated by extending a slot 77 from the suture lumen 78 to the surface of at least the distal body portion 76 as depicted in FIG. 13. In this variation, the suture 12 is fitted through the slot 77 when the suture assembly 10 is fitted to the suture delivery instrument 70 during assembly. It will be understood that the slot 77 may extend to the suture lumen 78 extending through the entire length of the suture instrument body 72.

It will also be understood that a similar laterally extending release slot 87 may extend from the snare cavity 88 through the length of the snare body 82 to facilitate releasing the suture 12 from the snare 80 after most of the suture 12 is drawn out of the snare cavity 88. The slot 87 may be formed in the snare body 82 extending from the suture lumen opening in snare distal end 84 proximally along snare cavity 88 and into the snare proximal end 86.

In a further variation of this embodiment of the invention, the suture proximal end 14 is formed into a suture loop 15 depicted in FIG. 14 supported within and/or by the membrane 90. The loop 15 is knotted with a slip-knot 17 proximal to the stored suture coil 19 that can be drawn tight about the suture distal end 16 when the tissue-penetrating barb 20 perforates the membrane 90. The perforated membrane 90 and/or the suture loop 15 detach from the snare proximal end 86. In this variation, the suture 12 is coiled into the suture coil 19 and is played out of the snare cavity 88 as described above and shown in FIG. 10, and a proximal portion of suture 12 can be passed laterally through the slot 87 as the suture loop 15 detaches from the snare proximal end 86. The tightening of the delivered suture assembly 10 is depicted in FIGS. 19 and 20, described below.

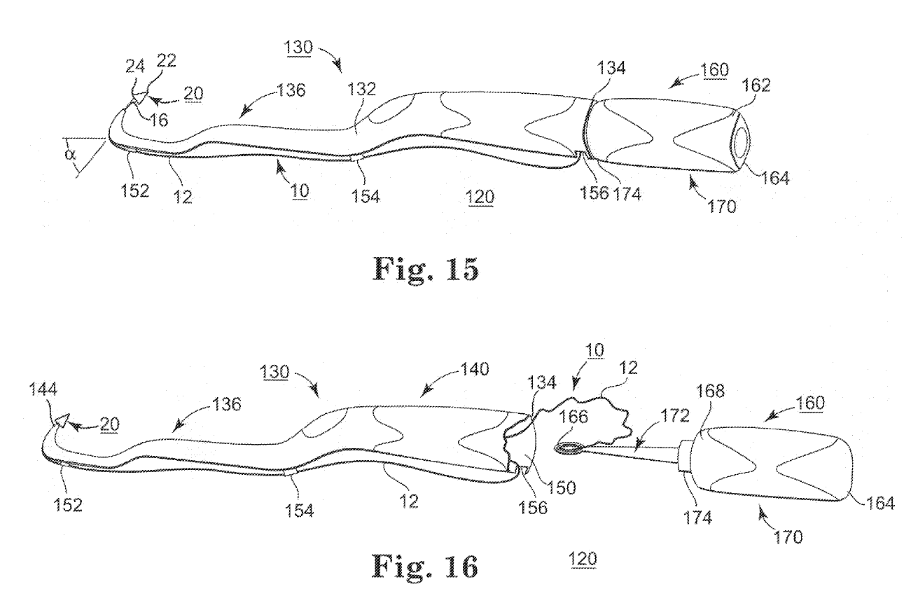

Turning to FIGS. 15-18, a variation of the suture delivery tool of FIGS. 9-12 is depicted that incorporates many of the features of the above-described embodiments. The suture delivery tool 120 combines a suture delivery instrument 130 and a suture snare 160 and also integrates, for ease of handling, any suture having a distal tissue-penetrating barb, e.g., the depicted suture assembly 10 having distal barb 20. The suture delivery instrument 130 detachably supports the suture snare 160 with the suture 12 extending therebetween as shown in FIG. 15. Again, the snare 160 supports a snare membrane 180 that provides the surgeon with positive audible and tactile feedback of capture of the tissue-penetrating barb 20 by the snare 160 when the snare 160 is detached as shown in FIG. 16. In this variation, the snare membrane 180 and a proximal length of the suture 12 are stored within a cavity 150 of the suture delivery instrument body 132 during fabrication, and are withdrawn from the instrument body cavity 150 after the distal barb 20 is passed through a ligament. The tightening of the delivered 12 about a ligament 100 is depicted in FIGS. 19 and 20.

The suture delivery instrument 130 comprises an elongated delivery instrument body 132 extending from a delivery instrument proximal end 134 to a delivery instrument distal end 144. The instrument body 132 comprises an elongated distal body portion 136 joined to a generally tubular proximal body portion 140. The distal body portion 136 is again preferably reduced in cross-section area from that of the proximal body portion 140 and is bent at an angle α that may be 90° or less to present the instrument body distal end 144 toward a side of the ligament. The distal body portion 136 has a shape configuration and cross-section area facilitating handling and providing sufficient column strength to be advanced trans-vaginally under visualization through the incision near the cervix and along one side of the uterosacral and cardinal ligaments.

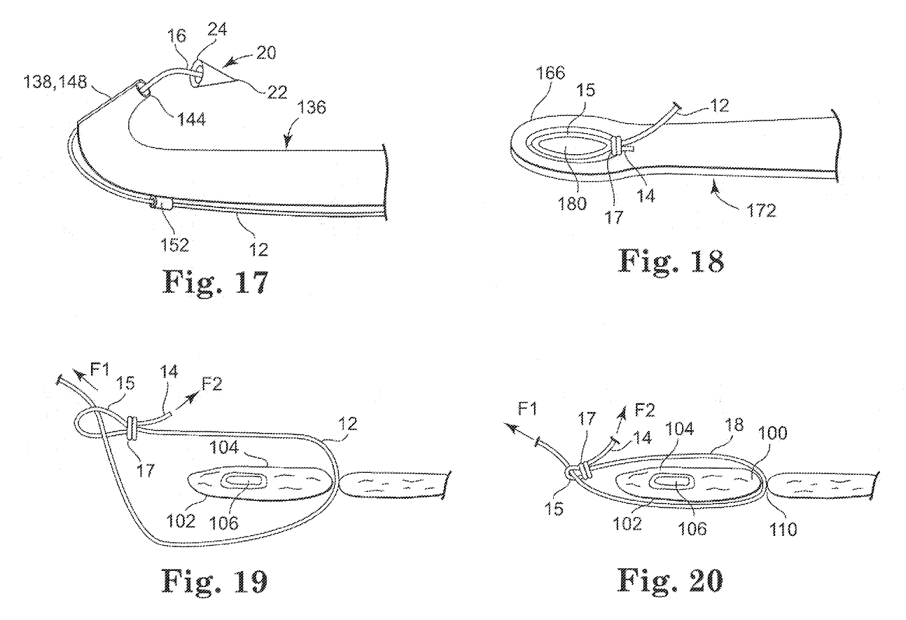

A suture lumen 138 with a side slot 148 extends along a distal section of distal body portion 136 as described above with respect to FIG. 13 and shown in greater detail in FIG. 17. The delivery instrument proximal end 134 is open to an instrument body cavity 150 extending substantially through the length of the proximal body portion 140. First and second, V-shaped, silicone rubber suture grippers 152 and 154 are provided along the length of distal body portion 136. A slot or notch 156 is formed through instrument body 132 into the instrument body cavity 150 proximate the instrument proximal end 134. Thus, a length of suture 12 can be extended through the notch 156 and distally alongside the instrument body 132 and be supported by suture grippers 152 and 154.

As noted above, one of the suture assemblies 10, 10′, 10″ or the like may be supported by the suture delivery instrument 130, e.g., suture assembly 10 depicted in FIGS. 15-18. At least a distal portion of suture 12 extends through the suture lumen 138 such that the tissue-penetrating barb 20 is disposed extending from the instrument body distal end 144. The enlarged shoulder 24 abuts the instrument body distal end 144 so that tissue-penetrating barb 20 cannot be drawn into suture lumen 138 and the sharp point 22 is extended distally from the instrument body distal end 144 to penetrate tissue.

The suture snare 160 also comprises a snare body 162 extending from a snare proximal end 164 to a ring-shaped snare distal end 166. A snare proximal segment 170 extends distally from the snare proximal end 164 and a snare distal segment 172 extends proximally from the snare distal end 166 to a mutual junction 174. The snare distal segment 172 is sized in diameter and length to be received within the instrument body cavity 150. The junction 174 is shaped is to be detachably coupled to the delivery instrument proximal end 134 employing any suitable friction or snap or threaded coupling mechanism or interlock upon full insertion of the snare distal segment 172 into instrument body cavity 150.

The snare distal end 166 supports a penetrable taut membrane 180 similar to and of the same material as the above-described membrane 90. As shown in detail in FIG. 18, the suture proximal end 14 is formed into a suture loop 15 supported within and/or by the membrane 180. The loop 15 is knotted with a slipknot 17 proximal to the stored suture coil 19 that can be drawn tight about the suture distal end 16 when the tissue-penetrating barb 20 perforates the membrane 180. The perforated membrane 180 and/or the suture loop 15 detach from the snare distal end 166. In this variation, the suture 12 is coiled into a suture coil (not shown) and is played out of the instrument body cavity 150 as shown in FIG. 16, and a proximal portion of suture 12 can be passed laterally through the slot 156 as the suture loop 15 detaches from the snare proximal end 166.

The suture delivery instrument 140 and snare 160 are provided in sterile packaging attached together and with the suture assembly 10 loaded as depicted in FIG. 15. The bulk of the suture 12 is loaded into the instrument body cavity 150 (shown in partial section in FIG. 16) during assembly of the suture 10, the snare 160, and suture delivery instrument 130. An intermediate length of the suture 12 can be drawn out of the body cavity 150 when the snare 160 is detached and separated apart from the suture delivery instrument 130 as shown in FIG. 10.

The instrument body 132 and the suture snare body 162 are especially shaped as depicted to have ergonomic manual gripping exterior surfaces that enhance handling and force transmission when assembled together as shown in FIG. 15. The bends and angles along the length of the instrument body 132, particularly in the distal portion 136 facilitate directing the instrument body distal end 144 along the ligament first side 102 to pass the tissue-penetrating barb 20 through the ligament 100. The passage is effected in the manner described above with respect to FIG. 11 with the instrument 130 and snare 160 assembled together as shown in FIG. 15. The snare 160 is detached as shown in FIG. 16, advanced along the second side of the ligament, and employed to snare the suture distal end 16 in the manner described above with respect to FIG. 12.

The ligation of the uterine artery 106 with the suture 12 having a distal suture loop 15 is shown in FIGS. 19 and 20. The suture 12 of suture assembly 10 extends through puncture 110 around and along the ligament sides 102 and 104, and the portions of the suture 12 near the proximal and distal suture ends 14 and 16 are made accessible to the surgeon. The tissue-penetrating barb 20 and the snare 80 are detached from the suture 12. The surgeon may then manually grasp or use suture gripping instruments to grip and tension the suture 12 applying forces F1 and F2 to pull loop 15 tight around suture 12. The further application of forces F1 and F2 results in compression of the ligament 100 and ligation of the uterine artery 106 to obstruct blood flow.

As noted above, the suture 12 is preferably formed of a resorbable or biodegradable material that degrades and is absorbed in the body after a time period sufficient to starve uterine fibroids or otherwise treat the uterus. The time period may or may not be long enough to cause permanent occlusion of the uterine artery at the ligated site.

All patents and publications referenced herein are hereby incorporated by reference in their entireties.

It will be understood that certain of the above-described structures, functions and operations of the above-described preferred embodiments are not necessary to practice the present invention and are included in the description simply for completeness of an exemplary embodiment or embodiments. It will also be understood that there may be other structures, functions and operations ancillary to the typical surgical procedures that are not disclosed and are not necessary to the practice of the present invention.

In addition, it will be understood that specifically described structures, functions and operations set forth in the above-referenced patents can be practiced in conjunction with the present invention, but they are not essential to its practice.

It is therefore to be understood, that within the scope of the appended claims, the invention may be practiced otherwise than as specifically described without actually departing from the spirit and scope of the present invention.

1. A method of occluding a blood vessel supported by a ligament of the human body, the ligament bounded by first and second opposed ligament sides, comprising:
providing a surgical suture assembly comprising a suture extending from a suture proximal end to a suture distal end and a tissue-penetrating barb secured to the suture distal end;
providing an elongated suture delivery instrument having a delivery instrument distal end adapted to engage and support the tissue-penetrating barb;
surgically accessing the first and second ligament sides;
advancing the suture delivery instrument along the first ligament side and the blood vessel;
manipulating the suture delivery instrument to pass the tissue-penetrating barb at the suture distal end through the ligament to the second ligament side;
providing an elongated suture snare having a snare element configured to engage and snare one of the tissue-penetrating barb and the suture substantially at or near the suture distal end;
advancing the suture snare along the second ligament side;
snaring one of the tissue-penetrating barbs and the suture substantially at or near the suture distal end;
retracting the suture snare and snared tissue-penetrating barb or suture along the second ligament side;
tightening the suture around the first and second sides of the ligament sufficiently to constrict and occlude the blood vessel; and
securing the tightened suture to maintain the occlusion of the blood vessel.

2. The method of claim 1, wherein:
the steps of providing the elongated suture delivery instrument and the elongated suture snare comprise providing the snare element at the suture delivery tool distal end; and
the step of advancing the suture snare comprises retracting the suture delivery instrument from the first ligament side and advancing the suture delivery instrument along the second ligament side and manipulating the snare element to engage and snare one of the tissue-penetrating barb and the suture substantially at or near the suture distal end.

3. The method of claim 1, wherein:
the steps of providing the elongated suture delivery instrument and the elongated suture snare comprise providing a detachable connection between the suture delivery instrument and the suture share; and
the step of advancing the suture snare comprises detaching the suture snare from the suture delivery instrument and advancing the suture snare along the second ligament side and manipulating the snare element to engage and snare one of the tissue-penetrating barb and the suture substantially at or near the suture distal end.

4. The method of claim 3, wherein:
the step of providing the surgical suture comprises:
supporting the suture proximal end by the suture snare;
storing a length of the suture within one of the suture snare and the suture delivery instrument; and
supporting the tissue-penetrating barb at the delivery instrument distal end; and
the step of advancing the suture snare comprises playing out the stored length of the suture as the suture snare is detached and advanced along the ligament second side.

5. The method of claim 4, wherein:
the step of providing the suture snare comprises supporting a penetrable membrane sized to be penetrable by the tissue-penetrating barb; and
the snaring step comprises manipulating the suture snare against the tissue-penetrating barb to effect penetration of the penetrable membrane and retention of the tissue-penetrating barb.

6. The method of claim 5, wherein:
the step of providing a suture comprises forming a suture loop proximate the suture proximal end;
the step of supporting the suture proximal end comprises releasably supporting the suture loop on the penetrable membrane disposed to enable passage of the tissue-penetrating barb through the suture loop; and
the steps of tightening and securing the suture comprise drawing the suture distal end through the suture loop and tightening the suture loop around the suture proximate the suture distal end.

7. The method of claim 4, wherein:
the step of providing the suture snare comprises supporting a penetrable membrane sized to be penetrable by the tissue-penetrating barb on the suture snare; and
the snaring step comprises manipulating the suture snare against the tissue-penetrating barb to effect penetration of the penetrable membrane and retention of the tissue-penetrating barb.

8. The method of claim 7, wherein:
the step of providing a suture comprises forming a suture loop proximate the suture proximal end;
the step of providing a suture snare comprises releasably supporting the suture loop on the penetrable membrane disposed to enable passage of the tissue-penetrating barb through the suture loop; and
the steps of tightening and securing the suture comprise drawing the suture distal end proximally through the suture loop and tightening the suture loop around the suture proximate the suture distal end.

9. The method of claim 4, wherein:
the step of providing a suture comprises forming a suture loop proximate the suture proximal end;
the step of providing a suture snare comprises releasably supporting the suture loop on the snare element disposed to enable passage of the tissue-penetrating barb through the suture loop; and
the steps of tightening and securing the suture comprise drawing the suture distal end proximally through the suture loop and tightening the suture loop around the suture proximate the suture distal end.

10. The method of claim 1, wherein:
the step of providing the surgical suture comprises:
supporting the suture proximal end by the suture snare;
storing a length of the suture within one of the suture snare and the suture delivery instrument; and
supporting the tissue-penetrating barb at the delivery instrument distal end; and
the step of advancing the suture snare comprises playing out the stored length of the suture as the suture snare is detached and advanced along the ligament second side.

11. The method of claim 10, wherein:
the step of providing the suture snare comprises supporting a penetrable membrane sized to be penetrable by the tissue-penetrating barb; and
the snaring step comprises manipulating the suture snare against the tissue-penetrating barb to effect penetration of the penetrable membrane and retention of the tissue-penetrating barb.

12. The method of claim 11, wherein:
the step of providing a suture comprises forming a suture loop proximate the suture proximal end;
the step of supporting the suture proximal end comprises releasably supporting the suture loop on the penetrable membrane disposed to enable passage of the tissue-penetrating barb through the suture loop; and
the steps of tightening and securing the suture comprise drawing the suture distal end proximally through the suture loop and tightening the suture loop around the suture proximate the suture distal end.

13. The method of claim 1, wherein:
the step of providing the suture snare comprises supporting a penetrable membrane sized to be penetrable by the tissue-penetrating barb; and
the snaring step comprises manipulating the suture snare against the tissue-penetrating barb to effect penetration of the penetrable membrane and retention of the tissue-penetrating barb.

14. The method of claim 13, wherein:
the step of providing a suture comprises forming a suture loop proximate the suture proximal end;
the step of providing a suture snare comprises releasably supporting the suture loop on the penetrable membrane disposed to enable passage of the tissue-penetrating barb through the suture loop; and
the steps of tightening and securing the suture comprise drawing the suture distal end through the suture loop and tightening the suture loop around the suture proximate the suture distal end.

15. The method of claim 1, wherein:
the step of providing a suture comprises forming a suture loop proximate the suture proximal end;
the step of providing a suture snare comprises releasably supporting the suture loop on the snare element disposed to enable passage of the tissue-penetrating barb through the suture loop; and
the steps of tightening and securing the suture comprise drawing the suture distal end through the suture loop and tightening the suture loop around the suture proximate the suture distal end.

16. Apparatus for occluding a blood vessel supported by a ligament of the human body, the ligament bounded by first and second opposed ligament sides and accessed surgically, the apparatus comprising:
a surgical suture assembly comprising a suture extending between a suture proximal end and a suture distal end and a tissue-penetrating barb secured to the suture distal end;
an elongated suture delivery instrument having a delivery instrument distal end adapted to engage and support the tissue-penetrating barb and adapted to be advanced along the first ligament side and the blood vessel and manipulated to pass the tissue-penetrating barb at the suture distal end through the ligament to the second ligament side; and
an elongated suture snare having a snare element configured to engage and snare one of the tissue-penetrating barb and the suture substantially at or near the suture distal end upon advancement of the suture snare along the second ligament side,
whereby one of the tissue-penetrating barb and the suture substantially at or near the suture distal end can be snared and retracted along the second ligament side to enable tightening and securing of the suture around the first and second sides of the ligament sufficiently to constrict and occlude the blood vessel.

17. The apparatus of claim 16, wherein the elongated suture delivery instrument and the elongated suture snare are combined to provide the snare element disposed at the suture delivery tool distal end,
whereby the suture delivery instrument distal end is retractable from the first ligament side and advanceable along the second ligament side to manipulate the snare element to engage and snare one of the tissue-penetrating barb and the suture substantially at or near the suture distal end.

18. The apparatus of claim 16, wherein the elongated suture delivery instrument and the elongated suture snare are configured to provide a detachable connection between the suture delivery instrument and the suture snare,
whereby the suture snare is detachable from the suture delivery instrument and advanceable along the second ligament side to manipulate the snare element to engage and snare one of the tissue-penetrating barb and the suture substantially at or near the suture distal end.

19. The apparatus of claim 18, wherein:
the suture proximal end is supported by the suture snare; and
a body cavity is formed in one of the suture snare and the suture delivery instrument that receives a length of the suture enabling playing out the stored length of the suture as the suture snare is detached from the suture delivery instrument and advanced along the ligament second side.

20. The apparatus of claim 19, wherein the suture snare comprises a penetrable membrane sized to be penetrable by the tissue-penetrating barb upon manipulation of the suture snare against the tissue-penetrating barb to effect penetration of the penetrable membrane and retention of the tissue-penetrating barb.

21. The apparatus of claim 20, wherein the suture comprises a suture loop proximate the suture proximal end releasably supported on the penetrable membrane disposed to enable passage of the tissue-penetrating barb through the suture loop,
whereby the suture distal end may be tightened and secured by drawing the suture proximally through the suture loop and tightening the suture loop around the suture proximate the suture distal end.

22. The apparatus of claim 20, wherein the suture snare comprises a penetrable membrane sized to be penetrable by the tissue-penetrating barb upon manipulation of the suture snare against the tissue-penetrating barb to effect penetration of the penetrable membrane and retention of the tissue-penetrating barb.

23. The apparatus of claim 22, wherein the suture comprises a suture loop proximate the suture proximal end releasably supported on the penetrable membrane disposed to enable passage of the tissue-penetrating barb through the suture loop,
whereby the suture distal end may be tightened and secured by drawing the suture through the suture loop and tightening the suture loop around the suture proximate the suture distal end.

24. The apparatus of claim 20, wherein the suture comprises a suture loop proximate the suture proximal end releaseably supported on the snare element and disposed to enable passage of the tissue-penetrating barb through the suture loop,
whereby the suture distal end may be tightened and secured by drawing the suture proximally through the suture loop and tightening the suture loop around the suture proximate the suture distal end.

25. The apparatus of claim 16, wherein:
the suture proximal end is supported by the suture snare; and
a body cavity is formed in one of the suture snare and the suture delivery instrument that receives a length of the suture enabling playing out the stored length of the suture as the suture snare is detached from the suture delivery instrument and advanced along the ligament second side.

26. The apparatus of claim 25, wherein the suture snare comprises a penetrable membrane sized to be penetrable by the tissue-penetrating barb upon manipulation of the suture snare against the tissue-penetrating barb to effect penetration of the penetrable membrane and retention of the tissue-penetrating barb.

27. The apparatus of claim 26, wherein the suture comprises a suture loop proximate the suture proximal end releaseably supported on the snare membrane and disposed to enable passage of the tissue-penetrating barb through the suture loop,
whereby the suture distal end may be tightened and secured by drawing the suture proximally through the suture loop and tightening the suture loop around the suture proximate the suture distal end.

28. The apparatus of claim 16, wherein the suture snare comprises a penetrable membrane sized to be penetrable by the tissue-penetrating barb upon manipulation of the suture snare against the tissue-penetrating barb to effect penetration of the penetrable membrane and retention of the tissue-penetrating barb.

29. The apparatus of claim 28, wherein the suture comprises a suture loop proximate the suture proximal end releaseably supported on the snare membrane and disposed to enable passage of the tissue-penetrating barb through the suture loop,
whereby the suture distal end may be tightened and secured by drawing the suture proximally through the suture loop and tightening the suture loop around the suture proximate the suture distal end.

30. The apparatus of claim 16, wherein the suture comprises a suture loop proximate the suture proximal end releaseably supported on the snare element and disposed to enable passage of the tissue-penetrating barb through the suture loop,
whereby the suture distal end may be tightened and secured by drawing the suture proximally through the suture loop and tightening the suture loop around the suture proximate the suture distal end.
